(19) United States
(12) Patent Application Publication
(10) Pub. No.: US 2007/0098786 A1
(43) Pub. Date: May 3, 2007

(54) ENTERIC VALPROIC ACID

(75) Inventors: Nachiappan Chidambaram, High Point, NC (US); Aqeel Fatmi, Greensboro, NC (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Banner Pharmacaps, Inc.

(21) Appl. No.: 11/548,607

(22) Filed: Oct. 11, 2006

Related U.S. Application Data
(63) Continuation-in-part of application No. 11/247,389, filed on Oct. 11, 2005.

(51) Int. Cl. A61K 9/64; A61K 31/19

(52) U.S. Cl. 424/456; 514/557

(57) ABSTRACT

An enteric valproic acid soft gelatin capsule, in which the enteric polymer is a component of the capsule shell rather than a coating, has been developed. The fill material comprises valproic acid or divalproex sodium and, optionally, one or more pharmaceutically acceptable excipients such as corn oil. The capsule shell is prepared from a mass comprising a film-forming polymer, an acid insoluble polymer, an aqueous solvent, and optionally a plasticizer. Suitable film-forming polymers include gelatin. Suitable acid-insoluble polymers include acrylic-acid/methacrylic acid copolymers. The acid-insoluble polymer is present in an amount from about 8% to about 20% by weight of the wet gel mass. The weight ratio of acid-insoluble polymer to film-forming polymer is from about 25% to about 50%. The aqueous solvent is water or an aqueous solution of alkalis such as ammonia or diethylene amine or hydroalcoholic solutions of the same. Suitable plasticizers include glycerin and triethylcitrate. The enteric soft gelatin capsule does not require an enteric coating and thus is not susceptible to the processing problems associated with enteric coated dosage forms. Enteric valproic acid soft gelatin capsules may be smaller in size and thus easier to swallow than currently available enteric coated tablets due to the presence of fewer ingredients, as well as smaller amounts of ingredients in the capsule shell.

### FIELD OF THE INVENTION

This invention is the field of pharmaceutical compositions, specifically an enteric valproic acid gelatin capsule formulation.

This application claims priority under 35 U.S.C. 120 to U.S.S.N. 11/247,389 filed Oct. 11, 2005.

### BACKGROUND OF THE INVENTION

Valproic Acid, or 2-propylpentanoic acid, and its salt and derivatives are used to treat absence seizures, complex partial seizures, mania, migraine headache prophylaxis, and behavior dyscontrol. Once in the body, valproic acid and its salts and derivatives are converted to valproate ion, which is responsible for the therapeutic effect. Valproic acid and its salt and derivatives are also known to cause significant side effects including gastrointestinal discomfort (nausea, indigestion, vomiting, diarrhea, and abdominal pain) which can decrease patient compliance.

Valproic acid and sodium valproate are difficult to formulate into solid oral dosage forms. Sodium valproate is extremely hygroscopic, often liquifying rapidly under ambient conditions. Valproic acid is an oily liquid at room temperature and thus not suitable for manufacturing solid dosage forms, e.g. tablets for oral administration.

Efforts have been made to address the problems associated with formulating valproic acid and sodium valproate into solid oral dosage forms. U.S. Pat. No. 5,017,613 to Aubert et al, describes a process for preparing a composition containing valproic acid in combination with valproate sodium. A mixture of valproic acid and ethylcellulose is prepared and valproate sodium is added to the mixture to form drug granules in the absence of any binder or granulating solvent. Precipitated silica is added to the granules before the granules are compressed into tablets. U.S. Pat. Nos. 5,212,326 and 4,988,731 to Meade describe divalproex sodium and its preparation. Divalproex sodium is a stable 1:1 ionic oligomer in which valproic acid forms coordinate bonds with the sodium of the sodium valproate salt.

Sustained release forms of divalproex sodium, valproic acid and its salts and derivatives have been developed in an effort to minimize the gastrointestinal side effects associated with these compounds. For example, U.S. Pat. No. 5,807,574 to Cheskin et al. describes a controlled release dosage form containing divalproex sodium and a process for its preparation. The process involves melting divalproex sodium and mixing it with a molten wax to form a divalproex sodium-wax composite. The drug-wax mixture is formulated into a capsule. U.S. Pat. No. 5,169,642 to Brinker et al. describes a sustained release dosage form containing granules of divalproex sodium, valproic acid or amides or esters or salts thereof and a polymeric viscosity agent. The drug is coated with a sustained release composition comprising specified portions of ethylcellulose or a methacrylic methylester, a plasticizer, and a detactifying agent.

Enteric-coated dosage forms are typically produced by a film coating process, where a thin film layer of an acid-insoluble (enteric) polymer is applied to the surface of a pre-manufactured dosage form, such as a tablet, and to a lesser extent hard and soft capsules. The enteric coating is sprayed as an aqueous or organic solution or suspension of one or more enteric polymers onto tumbling or moving tablets or capsules, followed by drying at elevated temperatures. Enteric dosage forms made by this coating method can suffer from various process-related problems that affect the performance and/or appearance of the coating. For example, “orange peel” surface formation, also known as surface roughness or mottling, may result. More seriously, coat integrity failure may occur, such as cracking or flaking off of the enteric polymer coating.

U.S. Pat. No. 5,068,110 to Fawzi et al. describes various currently marketed delayed-release tablets and capsules, including the delayed-release divalproex sodium tablets manufactured by Abbott Laboratories (Depakote® ER). Fawzu states that the stability of the enteric coated capsules is increased by applying thicker layer of the enteric coating, alone or in combination with hydroxypropyl cellulose or hydroxymethylcellulose.

All coating processes present inherent problems, including possible uneven distribution of the coating ingredients, which can occur under multivariate coated processes. These problems are common to all enteric dosage forms. However, the problems faced during the coating of gelatin or polysaccharide capsules are even more critical due to the delicate and heat sensitive nature of the soft elastic capsule shell. Both hard and soft capsules can undergo thermally induced agglomeration and distortion of the capsule shell. Moreover, the smoothness and elasticity of the capsule surface makes it difficult to form an intact adhering enteric coating. Moreover, the enteric coatings cause the loss of the normally shiny and clear appearance of gelatin capsule shells, which is a major reason for the popularity and acceptance of gelatin capsules. WO 2004/030658 to Banner Pharmacaps, Inc. describes a process and resulting enteric capsule which avoids these problems with most drugs by incorporating the enteric polymer into the gelatin, rather than onto the gelatin.

It is therefore an object of the present invention to provide an enteric valproic acid soft gelatin capsule dosage form which does not suffer from the processing limitations and poor stability associated with traditional enteric coated dosage forms.

It is another object of the present invention to provide an enteric valproic acid soft gelatin capsule dosage from which minimizes the gastrointestinal side effects associated with valproic acid.

It is yet another object of the present invention to provide an enteric valproic acid soft gelatin capsule dosage form which is smaller, uses fewer ingredients, and is therefore easier to swallow, than conventional enteric valproic acid dosage forms.

It is still another object of the present invention to provide a method of making an enteric valproic acid soft gelatin capsule dosage form which is more economical than other methods.

### SUMMARY OF THE INVENTION

An enteric valproic acid soft gelatin capsule, in which the enteric polymer is a component of the capsule shell rather than a coating, has been developed. The fill material comprises valproic acid or divalproex sodium and, optionally, one or more pharmaceutically acceptable excipients such as corn oil. The capsule shell is prepared from a mass comprising a film-forming polymer, an acid insoluble polymer, an aqueous solvent, and optionally a plasticizer. Suitable film-forming polymers include gelatin. Suitable acid-insoluble polymers include acrylic-acid/methacrylic acid copolymers. The acid-insoluble polymer is present in an amount from about 8% to about 20% by weight of the wet gel mass. The weight ratio of acid-insoluble polymer to film-forming polymer is from about 25% to about 50%. The aqueous solvent is water or an aqueous solution of alkalis such as ammonia or diethylene amine or hydroalcoholic solutions of the same. Suitable plasticizers include glycerin and triethylcitrate.

The enteric soft gelatin capsule does not require an enteric coating and thus is not susceptible to the processing problems associated with enteric coated dosage forms. Enteric valproic acid soft gelatin capsules can be smaller in size and thus easier to swallow than currently available enteric coated tablets due to the presence of fewer ingredients, as well as smaller amounts of ingredients, in the capsule shell. In addition, the cost of manufacture due to the fewer processing steps and ingredients, is significantly less than with other methods.

### BRIEF DESCRIPTION OF THE DRAWINGS

FIG. 1 is a graph of the mean serum concentration of valproic acid from 0 to 72 hours after dose administration of Valproic Acid Enteric 500 mg Softgel Capsules under fasting and non-fasting conditions, and Depakote© Delayed-Release 500 mg Tablets under fasting conditions.

FIG. 2 is a graph of the mean serum concentration of valproic acid from 0 to 72 hours after dose administration of Valproic Acid Enteric 500 mg Softgel Capsules and Depakote© Delayed-Release 500 mg Tablets under non-fasting conditions.

FIG. 3 is a graph of the estimated time to steady state for Valproic Acid Enteric 500 mg Softgel Capsules and Depakote© Delayed-Release 500 mg Tablets based on pharmacokinetic data.

### DETAILED DESCRIPTION OF THE INVENTION

I. Composition

A. Capsule Fill

1. Valproic Acid

Valproic acid, or 2-propylpentanoic acid, and its salts and derivatives are compounds which have been used to treat absence seizures, complex partial seizures, mania, migraine headaches prophylaxis, and behavior dyscontrol. Valproic acid (available from Sifa Ltd., Shannon, Ireland; Interchem and Katwijk Chemie, the Netherlands; and Generichem) is an oily liquid at room temperature. Valproic acid is colorless and has a characteristic odor. It is slightly soluble in water (1.3 mg/mL) and very soluble in organic solvents. Valproic acid can be used neat or as a solution. The concentration of valproic acid in the fill material is from about 25% to about 100% by weight of the fill material. In the preferred embodiment, divalproex sodium is present in the fill at a concentration of about 40% by weight of the fill. Total dosage per capsule is typically 250 mg, although 125 mg and 500 mg sizes are also useful.

Divalproex sodium can also be used in the formulation of enteric soft gelatin capsules. Divalproex sodium is a 1:1 molar ratio oligomer of free valproic acid and sodium valproate. Divalproex sodium (available from SST Crop., New Jersey) is a white, crystalline powder, which is soluble in water and alcoholic solvents such as methanol and ethanol, as well as organic solvents such as cyclohexane.

2. Excipients

The capsule fill may be prepared using a pharmaceutically acceptable carrier composed of materials that are considered safe and effective and may be administered to an individual without causing undesirable biological side effects or unwanted interactions. The carrier consists of all components present in the pharmaceutical formulation other than the active ingredient or ingredients. As generally used herein “carrier” includes, but is not limited to, plasticizers, crystallization inhibitors, wetting agents, bulk filling agents, solubilizers, bioavailability enhancers, solvents, pH-adjusting agents and combinations thereof.

Suitable excipients include one or more solubilizers such as soybean oil, rapeseed oil, safflower oil, corn oil, olive oil, castor oil, oleic acid, medium chain triglycerides, mono- and diglycerides (available from Abitec Corp., Columbus, Ohio, under the tradename Capmul®), medium chain triglyceride esters (available from Abitec Corp., Columbus, Ohio, under the tradename Captex®), medium chain partial triglycerides (available from Sasol under the tradename Imwitor®), corn oil-PEG 6 complex (available from Gattefosse S.A., Saint Priest, France under the tradename Labrasol®), propylene glycol monolaurate (lauraglycol), long chain partial glycerides (available from Gattefosse S.A., Saint Priest, France, under the tradename Maisine®), sorbitan monooleate (available from ICI under the tradename Span®), polysorbates (available from ICI under the tradename Tween®), ethoxylated castor oil (cremophors), bees wax, hydrogenated soybean oil, partially hydrogenated soybean oil, and acetylated triglycerides. In a preferred embodiment, the solubilizer is corn oil.

B. Capsule Shell

The capsule shell is prepared from a gelatin mass comprising a film-forming polymer, an acid-insoluble polymer which is present in an amount making the capsule resistant to the acid within the stomach, an aqueous solvent, and optionally, one or more plasticizers and/or colorants. Other suitable shell additives including opacifiers, colorants, humectants, preservatives, flavorings, and buffering salts and acids. Enteric capsule shells and a method of making the capsule shell are described in WO 2004/030658 to Banner Pharmacaps, Inc.

1. Film-Forming Polymers

Exemplary film-forming polymers can be of natural or synthetic origin. Natural film-forming polymers include gelatin and gelatin-like polymers. Other suitable natural film-forming polymers include shellac, alginates, pectin, and zeins. Synthetic film-forming polymers include hydroxypropyl methyl cellulose, methyl cellulose, hydroxypropyl methyl cellulose acetate phthalate, and acrylates such as poly(meth)acrylate. The weight ratio of acid-insoluble polymer to film-forming polymer is from about 15% to about 50%. In one embodiment, the film forming polymer is gelatin.

2. Acid-Insoluble Polymers

Exemplary acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers (available under the tradename EUDRAGIT® from Rohm America Inc., Piscataway, N.J. as a powder or a 30% aqueous dispersion; or under the tradename EASTACRYL®, from Eastman Chemical Co., Kingsport, Tenn., as a 30% dispersion). In one embodiment, the acid-insoluble polymer is EUDRAGIT® L100, which is a methacrylic acid/methacrylic acid methyl ester copolymer. The acid-insoluble polymer is present in an amount from about 8% to about 20% by weight of the wet gelatin mass. The weight ratio of acid-insoluble polymer to film-forming polymer is from about 15% to about 50%.

3. Aqueous Solvent

Exemplary aqueous solvents include water or aqueous solutions of alkalis such as ammonia, sodium hydroxide, potassium hydroxide, ethylene diamine, hydroxylamine, tri-ethanol amine, or hydroalcoholic solutions of the same. The alkali can be adjusted such that the final pH of the gelatin mass is less than or equal to 9.0, preferably less than or equal to 8.5, more preferably less than or equal to 8.0. In one embodiment, the alkali is a volatile alkali such as ammonia or ethylene diamine.

4. Plasticizers

Exemplary plasticizers include glycerol, glycerin, sorbitol, polyethylene glycol, citric acid, citric acid esters such as triethylcitrate, polyalcohols with 3-6 carbons and combinations thereof. The plasticizer to polymer (film forming polymer plus acid-insoluble polymer) ratio is from about 10% to about 50% of the polymer weight.

II. Method of Manufacture

A. Capsule Fill

Valproic acid or divalproex is dispensed into a suitable container and, optionally, mixed with a diluting vehicle such as corn oil. The fill is deaerated prior to encapsulation in a soft gelatin capsule.

B. Capsule Shell

A method of making the capsule shell is described in WO 2004/030658 to Banner Pharmacaps, Inc. The enteric gelatin mass can be manufactured by preparing an aqueous solution comprising a film-forming, water soluble polymer and an acid-insoluble polymer and mixing the solution with one or more appropriate plasticizers to form a gelatin mass. Alternatively, the enteric gelatin mass can be prepared by using a ready-made aqueous dispersion of the acid-insoluble polymer by adding alkaline materials such as ammonium, sodium, or potassium hydroxides or other alkalis that will cause the acid-insoluble polymer to dissolve. The plasticizer-wetted, film-forming polymer can then be mixed with the solution of the acid-insoluble polymer. The gelatin mass can also be prepared by dissolving the acid-insoluble polymer or polymers in the form of salts of the above-mentioned bases or alkalis directly in water and mixing the solution with the plasticizer-wetted, film-forming polymer. The gelatin mass is cast into films or ribbons using heat controlled drums or surfaces. The fill material is encapsulated in a soft gelatin capsule using a rotary die. The capsules are dried under controlled conditions of temperature and humidity. The final moisture content of the shell composition is from about 2% to about 10% by weight of the capsule shell, preferably from about 4% to about 8% by weight by weight of the capsule shell.

III. Method of Use

Enteric valproic acid soft gelatin capsules can be used to administer valproic acid or divalproex sodium to a patient in need thereof. In the preferred embodiment the capsule contains dose equivalents of 125 mg, 250 mg, or 500 mg.

The data in the following examples demonstrates that it is possible to make capsules or soft gelatin capsules that release valproic acid to produce the following pharmacokinetic profiles:

wherein the valproic acid is released following oral administration to a fasting individual to produce a Cₘₐₓ between approximately 37.6 and 72.5 mg valproic acid/ml blood with a Tₘₐₓ of between 1 and 4 hours, more preferably wherein the Cₘₐₓ is between 42.3 and 67.5 mg valproic acid/ml blood with a Tₘₐₓ of between 1.35 and 3 hours; and

wherein the valproic acid is released following oral administration to a non-fasting individual to produce a Cₘₐₓ between 27.2 and 58.64 mg valproic acid/ml blood with a Tₘₐₓ of between 3 and 9 hour, more preferably wherein the Cₘₐₓ is between 31 and 53.8 mg valproic acid/ml blood with a Tₘₐₓ of between 3 and 9 hours.

Although described in the examples with reference to specific enteric polymer containing soft gelatin capsules, those skilled in the art will recognize that other capsules or soft gelatin capsules can be similarly prepared to achieve equivalent pharmacokinetic drug profiles.

### EXAMPLES

### Example 1

### Enteric Gelatin Mass

A gelatin mass was made according to the formula below.

| | |
|---|---|
| Gelatin | 28.00% |
| Eudragit ® L100 | 9.00% |
| Glycerin | 15.4% |
| Triethyl citrate | 0.90% |
| Ammonium hydroxide | 0.05% |
| Water | 46.65% |

The acid insoluble polymer (Eudragit® L 100) was dissolved in an aqueous alkali solution (water and ammonium hydroxide). The film-forming polymer (gelatin), and any plasticizers (glycerin), colorants, or other shell additives were added to the acid insoluble polymer solution and the mixture was cooked via a hot-melt process. The water content of the gelatin mass was adjusted to the indicated level. The gelatin mass was deaerated and dropped into a receiver. The dropped gelatin mass was held in the receivers at a temperature between 110 and 140° F. until encapsulation.

### Example 2

### Enteric Soft Capsules with Valproic Acid Fill

Enteric soft capsules were prepared using a conventional rotary die process. The enteric gelatin mass from Example 1 was cast as a thin ribbon. The appropriate fill mass was pumped into each die cavity in order to provide the appropriate fill weight. After the die cavities were filled, the ribbon was sealed to form capsules of the desired shape and size. The capsules were dried initially in a tumble dryer and then dried on trays in a drying tunnel until the desired hardness was achieved. The dried capsules were then inspected, sized, printed, polished and packaged.

### Example 3

### Relative Bioavailability Study of Valproic Acid Enteric 500 mg Softgel Capsules Under Fasting Conditions

The pharmacokinetic parameters of Valproic Acid Enteric 500 mg Softgel capsules was compared to that of a reference compound. Depakote® Delayed-Release Tablets (500 mg).

The objective of this randomized, single-dose, three-way crossover study was to compare, under fasting conditions, the relative bioavailability (rate and extent of absorption) of Valproic Acid Enteric 500 mg Softgel to that of an equivalent dose of Depakote® Delayed-Release Tablets, when administered to healthy subjects.

Material and Methods

Thirty-six healthy adults participated in the comparison between Valproic Acid Enteric 500 mg Softgel and Depakote® Delayed-Release Tablets. All 36 subjects completed the study. On Day 1, following an overnight fast of at least 10 hours, subjects received a single, oral dose (1×500 mg) of either the test Valproic Acid Enteric 500 mg Softgel or the reference Depakote® Delayed-Release Tablets 500 mg with 240 mL ambient temperature water, as per the randomization scheme.

During each study period, 21 blood samples were collected (7 mL each) from each subject by direct venipuncture using pre-labeled vacutainers without anticoagulant. Blood samples were collected within 1 hour prior to dose administration (0 hour) and at 0.5, 1, 1.5, 2, 2.5, 3, 3.5, 4, 4.5, 5, 5.5, 6, 7, 8, 12, 16, 24, 36, 48, and 72 hours after dose administration.

Approximately 441 mL of blood was collected from each subject for pharmacokinetic samples over the course of the study. Upon completion of the clinical study, the serum samples were transferred to the PRACS Institute, Ltd. Bioanalytical Laboratory for sample analysis.

Serum concentration data from all 36 subjects were used in the pharmacokinetic and statistical analysis. The lower limit of quantitation for valproic acid was 2.00 μg/mL. For statistical analysis, subject sample values below the lower limit of quantitation (BLQ) were reported as zero.

The following pharmacokinetic parameters were calculated for each subject and period: peak concentration in plasma (Cₘₐₓ), time to peak concentration (Tₘₐₓ), elimination rate constant (kₑ), terminal half-life (t_{1/2}), area under the concentration-time curve calculated according to the linear trapezoidal rule (AUC₀₋ₜ), and area under the plasma concentration time curve from time-zero extrapolated to infinity (AUC_{0-∞).}

An analysis of variance (ANOVA) was performed on each of the pharmacokinetic parameters using SAS® software. The ANOVA model containing factors for sequence of products, subjects within sequence, periods and products was utilized in comparing the effects between the test and reference products. Differences were declared statistically significant at the 5% level.

A 90% confidence interval about the ratio of the mean test value to mean reference value was calculated for all of the pharmacokinetic parameters for each test product. The calculations for the confidence intervals used the least squares means (LSMEANS) and the standard error of the estimate, both generated by the SAS® software. The ratio of the geometric means for the In-transformed data and the corresponding 90% confidence intervals were calculated for AUC₀₋ₜ, AUC_{0-∞}, and Cₘₐₓ, as well. The statistical analysis was done using SAS®, Version 8.2 for Windows, using code based on Chow and Liu pp. 559-562.

Results

Table 1 shows both the non-transformed and the In-transformed data for the calculated pharmacokinetic parameters for Depakote® Delayed-Release Tablets (Treatment A) and Valproic Acid Enteric Softgel capsules (Treatment C). Table 1 also shows the statistical analysis of the non-transformed and the In-transformed data.

The 90% confidence intervals about the ratio of Treatment A (Test Product Fasting) geometric mean to Treatment C (Reference Product Fasting) geometric mean are within the 80% and 125% limits for the pharmacokinetic parameters Cₘₐₓ, AUC₀₋ₜ, and AUC_{0-∞} of the In-transformed data.

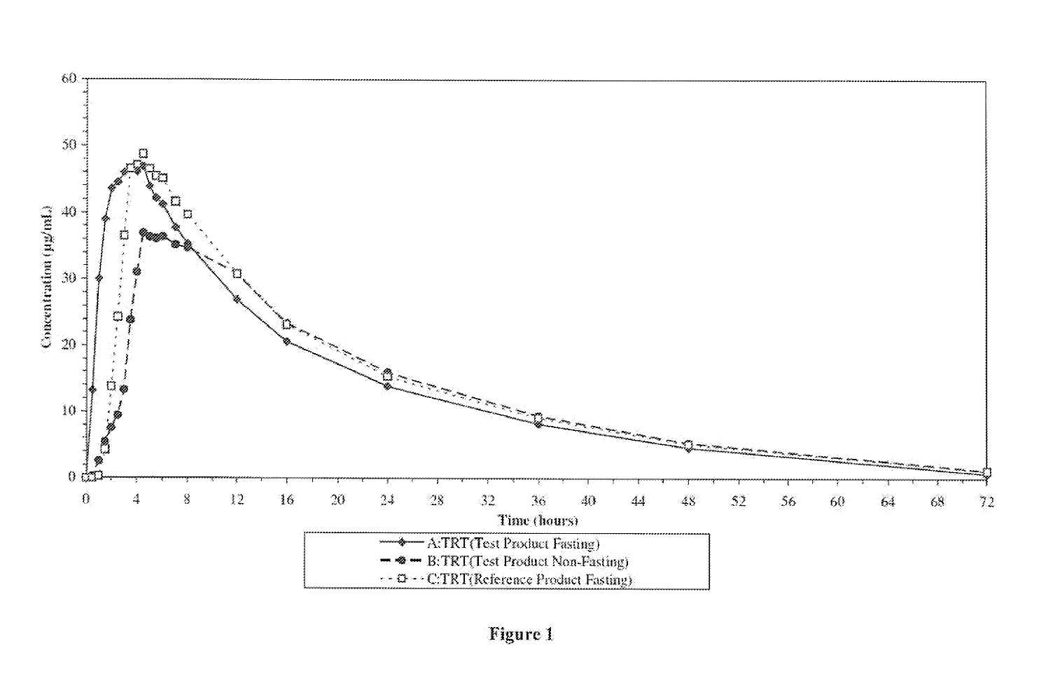

FIG. 1 shows the mean serum concentration of valproic acid from 0 to 72 hours after dose administration for Treatment A (Test Product Fasting) and Treatment C (Reference Product Fasting).

The results of this study indicate bioequivalence between the test Valproic Acid Enteric 500 mg Softgel and the reference Depakote® Delayed-Release Tablets 500 mg when administered under fasting conditions.

TABLE 1
Ln- and Non-Transformed Pharmacokinetic Parameters of Valproic Acid
After Oral Administration and Statistical Analysis
Treatment A (Test Product Fasting) vs. Treatment C (Reference Product Fasting)
N = 36
Ln-Transformed Data

| | | | | | | | 90% |
|---|---|---|---|---|---|---|---|
| | | | | | | | Confidence |
| | Least Squares Mean | | Geometric Mean | | | | Interval |
| PK | Treatment | Treatment | Treatment | Treatment | | Mean Square | (Lower Limit, |
| Variable | A | C | A | C | % Ratio | Error | Upper Limit) |
| Cₘₐₓ | 3.981 | 4.009 | 53.58 | 55.10 | 97.24 | 0.01210 | (93.13, 101.54) |
| AUC₀₋ₜ | 6.787 | 6.824 | 886.52 | 919.81 | 96.38 | 0.00295 | (94.35, 98.46) |
| AUC_{0-∞} | 6.877 | 6.908 | 969.52 | 1000.45 | 96.91 | 0.00272 | (94.94, 98.92) |

Non-Transformed Data

| | | | | | 90% |
|---|---|---|---|---|---|
| | | | | | Confidence |
| | Least Squares Mean | | | | Interval |
| Pk | Treatment | Treatment | | Mean Square | (Lower Limit, |
| Variable | A | C | % Ratio | Error | Upper Limit) |
| Cₘₐₓ | 53.98 | 55.61 | 97.06 | 25.63 | (93.49, 100.64) |
| AUC₀₋ₜ | 905.97 | 936.03 | 96.79 | 2318.10 | (94.77, 98.81) |
| AUC_{0-∞} | 989.01 | 1018.00 | 97.15 | 2483.63 | (95.23, 99.08) |
| Tₘₐₓ | 2.32 | 3.69 | 62.80 | 2.9737 | (44.44, 81.15) |
| kₑ | 0.0476 | 0.0473 | 100.58 | 0.00001 | (97.93, 103.23) |
| t_{1/2} | 15.07 | 15.32 | 98.41 | 0.9780 | (95.87, 100.94) |

Geometric means are based on least squares means of ln-transformed values.

### Example 4

### Relative Bioavailability Study of Valproic Acid Enteric 500 mg Softgel Capsules Under Fed Conditions

The objective of this randomized, single-dose, three-way crossover study was to compare the relative bioavailability (rate and extent of absorption) of Valproic Acid Enteric 500 mg Softgel under fasting and non-fasting conditions, when administered to healthy subjects. To determine the food effects for Valproic Acid Enteric Softgel, the pharmacokinetic data under fasting conditions was used as a reference. The same thirty-six subjects from Example 3 were enrolled in the food effect study.

Materials and Methods

All thirty-six enrolled subjects completed the study. For those subjects to be dosed under non-fasting conditions, a standardized, high fat breakfast was served 30 minutes prior to dose administration, as per the randomization. Thirty minutes after starting the standardized, high fat breakfast, subjects received a single, oral dose (1×500 mg) of the test Valproic Acid Enteric 500 mg Softgel with 240 mL of ambient temperature water. All subjects fasted for at least 4.25 hours after dosing. There was at least a seven day washout between study periods. Blood sample were taken and analyzed as described in Example 3.

Table 2 shows both the non-transformed and the In-transformed data for the calculated pharmacokinetic parameters for Valproic Acid Enteric Softgel capsules under fasting conditions (Treatment A) and Valproic Acid Enteric Softgel capsules under fed (non-fasting) conditions (Treatment B). Table 2 also shows the statistical analysis of the non-transformed and the In-transformed data.

The 90% confidence intervals about the ratio of Treatment A (Test Product Fasting) geometric mean to Treatment B (Test Product Non-Fasting) geometric mean are within the 80% and 125% limits for the pharmacokinetic parameters AUC₀₋ₜ, and AUC_{0-∞}, but not for Cₘₐₓ, of the In-transformed data.

TABLE 2
Ln- and Non-Transformed Pharmacokinetic Parameters of Valproic Acid
After Oral Administration and Statistical Analysis
Treatment B (Test Product Non-Fasting) vs. Treatment A (Test Product Fasting)
N = 36
Ln-Transformed Data

| | | | | | | | 90% |
|---|---|---|---|---|---|---|---|
| | | | | | | | Confidence |
| | Least Squares Mean | | Geometric Mean | | | | Interval |
| PK | Treatment | Treatment | Treatment | Treatment | | Mean Square | (Lower Limit, |
| Variable | B | A | B | A | % Ratio | Error | Upper Limit) |
| Cₘₐₓ | 3.714 | 3.981 | 41.02 | 53.58 | 76.56 | 0.01210 | (73.32, 79.94) |
| AUC₀₋ₜ | 6.745 | 6.787 | 849.44 | 886.52 | 95.82 | 0.00295 | (93.79, 97.88) |
| AUC_{0-∞} | 6.833 | 6.877 | 929.53 | 969.52 | 95.88 | 0.00272 | (93.93, 97.86) |

Non-Transformed Data

| | | | | | 90% |
|---|---|---|---|---|---|
| | | | | | Confidence |
| | Least Squares Mean | | | | Interval |
| PK | Treatment | Treatment | | Mean Square | (Lower Limit, |
| Variable | B | A | % Ratio | Error | Upper Limit) |
| Cₘₐₓ | 41.70 | 53.98 | 77.25 | 25.63 | (73.56, 80.93) |
| AUC₀₋ₜ | 869.15 | 905.97 | 95.94 | 2318.10 | (93.85, 98.02) |
| AUC_{0-∞} | 950.62 | 989.01 | 96.12 | 2483.63 | (94.14, 98.1) |
| Tₘₐₓ | 6.09 | 2.32 | 262.88 | 2.9737 | (233.65, 292.1) |
| kₑ | 0.0479 | 0.0476 | 100.60 | 0.00001 | (97.96, 103.24) |
| t_{1/2} | 13.04 | 15.07 | 99.77 | 0.9780 | (97.19, 102.35) |

Geometric means are based on least squares means of ln-transformed values.

FIG. 1 shows the mean serum concentration of valproic acid from 0 to 72 hours after dose administration for Treatment A (Test Product Fasting) and Treatment B (Test Product Non-Fasting).

The administration of Valproic Acid 500 mg Enteric Softgel capsules with food significantly decreased the In-transformed Cₘₐₓ (23.44%). However, food did not significantly decrease the In-transformed ACU₀₋ₜ (4.18%) and In-transformed AUC_{0-∞} (4.12%). Thus, administration of Valproic Acid Enteric 500 mg Softgel under non-fasting conditions did not affect the extent of absorption.

### Example 5

### Relative Bioavailability Study of Valproic Acid Enteric 500 mg Softgel Capsules Under Non-Fasting Conditions

An interview by Banner Pharmacaps, Inc. of physicians (N=24) indicated that a majority of their patients take Depakote© with food. Therefore, a randomized, two-way crossover design was used to compare the relative bioavailability (rate and extent of absorption) of Valproic Acid 500 mg Capsules with the reference compound, Dekapote© 500 mg Delayed-Release Tablets, under non-fasting conditions.

Materials And Methods

Six healthy subjects were used in this study. A single oral dose was administered to subjects on two separate occasions under non-fasting conditions with a 7 day washout between doses. Food and fluid intake were controlled during each confinement period.

Serum concentrations of valproic acid were determined by the bioanalytical laboratory of PRACS Institute, Ltd. Data from all six subjects was used for pharmacokinetic and statistical analysis. The pharmacokinetic parameters that were calculated were the same as for Examples 3 and 4. Actual times were used in the calculation of pharmacokinetic parameters.

Results

Table 3 shows the In-transformed data for the calculated pharmacokinetic parameters, Cₘₐₓ, AUC₀₋ₜ, and AUC_{0-∞}, for Valproic Acid Enteric Softgel Capsules (Test Product) and Depakote© Delayed-Release Tablets (Reference Product). Table 3 also shows the statistical analysis of the In-transformed data.

TABLE 3
Ln-Transformed Pharmacokinetic Parameters of Valproic Acid
After Oral Administration and Statistical Analysis

| | Ln- | Ln- | Ln- |
|---|---|---|---|
| | Transformed | Transformed | Transformed |
| Valproic Acid | Cₘₐₓ | AUC₀₋ₜ | AUC_{0-∞} |
| Test Product | 42.40 | 804.19 | 879.12 |
| Geometric Mean | | | |
| Reference Product | 50.49 | 856.91 | 922.54 |
| Geometric Mean | | | |
| % Ratio | 83.97 | 93.85 | 95.29 |
| 90% Confidence | (74.45, 94.72) | (88.78, 99.21) | (92.03, 98.67) |
| Intervals | | | |

Table 4 shows the non-transformed data for the calculated pharmacokinetic parameters, Cₘₐₓ, AUC₀₋ₜ, and AUC_{0-∞}, for Valproic Acid Enteric Softgel Capsules (Test Product) and Depakote© Delayed-Release Tablets (Reference Product). Table 4 also shows the statistical analysis of the non-transformed data.

TABLE 4
Non-Transformed Pharmacokinetic Parameters of Valproic Acid
After Oral Administration and Statistical Analysis

| Valproic Acid | Cₘₐₓ | AUC₀₋ₜ | AUC_{0-∞} |
|---|---|---|---|
| Test Product | 42.96 | 815.73 | 887.98 |
| Geometric Mean | | | |
| Reference Product | 51.26 | 869.81 | 932.21 |
| Geometric Mean | | | |
| % Ratio | 83.81 | 93.78 | 95.26 |
| 90% Confidence | (74.34, 93.28) | (88.18, 99.39) | (91.56, 98.95) |
| Intervals | | | |

Table 5 shows the non-transformed data for the calculated pharmacokinetic parameters, Tₘₐₓ, kₑ, and t_{1/2}, for Valproic Acid Enteric Softgel Capsules (Test Product) and Depakote© Delayed-Release Tablets (Reference Product). Table 5 also shows the statistical analysis of the non-transformed data.

TABLE 5
Non-Transformed Pharmacokinetic Parameters of Valproic Acid
After Oral Administration and Statistical Analysis

| Valproic Acid | Tₘₐₓ | kₑ | t_{1/2} |
|---|---|---|---|
| Test Product | 5.00 | 0.0464 | 15.11 |
| Least Squares Mean | | | |
| Reference Product | 10.67 | 869.81 | 932.21 |
| Least Squares Mean | | | |
| % Ratio | 46.88 | 93.78 | 95.26 |
| 90% Confidence | (24.28, 69.47) | (87.56, 100.81) | (99.44, 113.48) |
| intervals | | | |

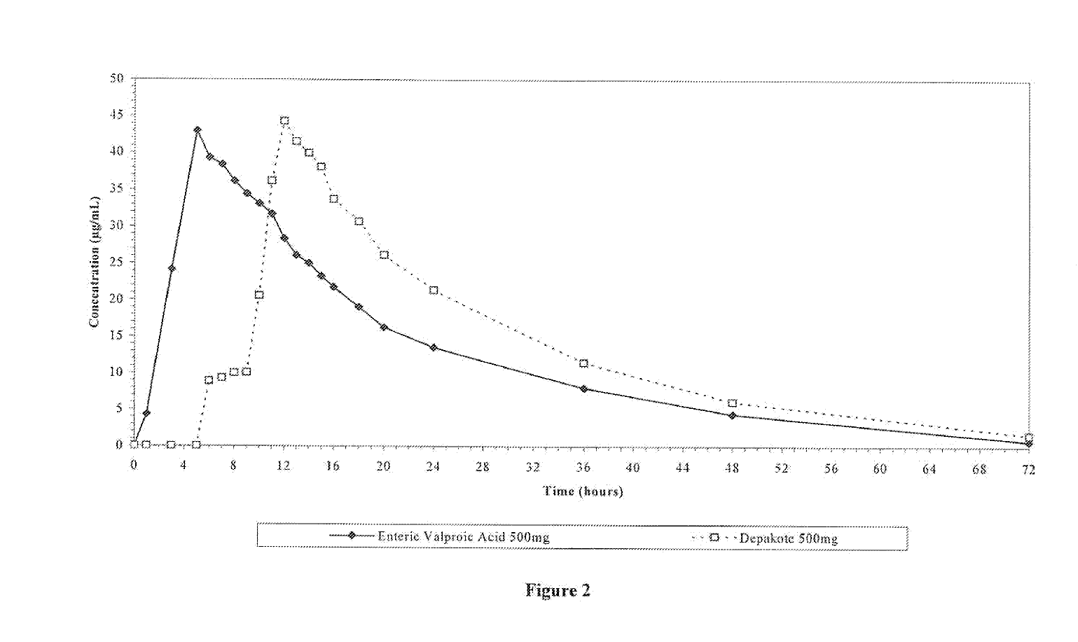

FIG. 2 shows the mean serum concentrations of valproic acid among subjects at each time point tested from 0 to 72 hours after dose administration of Valproic Acid Enteric Softgel Capsules (Enteric Valproic Acid 500 mg) and Depakote© Delayed-Release Tablets (Depakote 500 mg).

The results of this study indicate near equivalence of Valproic Acid Enteric Softgel Capsules and Depakote© Delayed-Release Tablets under non-fasting conditions with respect to the pharmacokinetic parameters Cₘₐₓ, AUC₀₋ₜ, and AUC_{0-∞}. However, Valproic Acid Enteric Softgel Capsules induced a significantly lower Tₘₐₓ, relative to Depakote© Delayed-Release Tablets, indicating a faster onset of action under non-fasting conditions.

### Example 6

### Relative Estimated Time to Steady State of Valproic Acid 500 mg Capsules

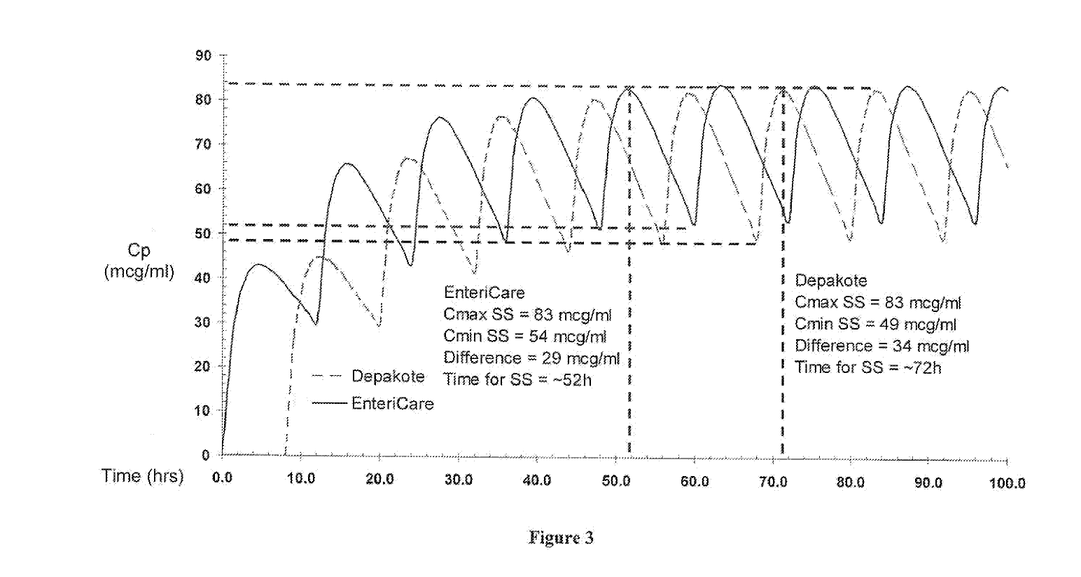

Time to steady state for Valproic Acid 500 mg Capsules and Depakote© Delayed-Release Tablets was estimated based on pharmacokinetic data from Example 5, as shown in FIG. 3. This estimation predicts that Valproic Acid 500 mg Capsules may attain steady state in 52 hours, while Depakote© Delayed-Release Tablets may attain steady state in 72 hours. Thus, Valproic Acid 500 mg Capsules are predicted to reach steady state 25% faster than Depakote© Delayed-Release Tablets.

It is understood that the disclosed invention is not limited to the particular methodology, protocols, and reagents described as these may vary. Unless defined otherwise, all technical and scientific terms used herein have the same meanings as commonly understood by one of skill in the art to which the disclosed invention belongs.

We claim:

1. An enteric valproic acid soft gelatin capsule comprising:
(a) a fill material comprising valproic acid or divalproex sodium; and
(b) a capsule shell comprising a gelatin mass, film-forming water soluble polymer, an acid-insoluble polymer, and an aqueous solvent.

2. The capsule of claim 1, wherein valproic acid is present in an amount from about 25% to about 100% by weight of the fill.

3. The capsule of claim 1, wherein the concentration of divalproex sodium is from about 25% to about 100% by weight of the fill, preferably 40%.

4. The capsule of claim 1 wherein the capsule contains a dosage of valproic acid, divalproex sodium, or mixture thereof selected from the group consisting of 125 mg, 250 mg, and 500 mg.

5. The capsule of claim 1 wherein the shell comprises additional components selected from the group consisting of plasticizers, coloring agents, opacifiers, humectants, preservatives, flavorings, and buffering salts and acids.

6. The capsule of claim 1, wherein the fill material further comprises one or more pharmaceutically acceptable excipients.

7. The capsule of claim 6, wherein the one or more excipients is selected from the group consisting of crystallization inhibitors, wetting agents, bulk filling agents, solubilizers, bioavailability enhancers, solvents, pH-adjusting agents, dyes, preservatives, solvents, surfactants, and combinations thereof.

8. The capsule of claim 6 wherein the excipient is a solubilizer selected from the group consisting of soybean oil, rapeseed oil, safflower oil, corn oil, olive oil, castor oil, oleic acid, medium chain triglycerides, mono- and diglycerides, medium chain triglyceride esters, medium chain partial triglycerides, sorbitan monooleate, polysorbates, ethoxylated castor oil, bees wax, hydrogenated soybean oil, partially hydrogenated soybean oil, and acetylated triglycerides.

9. The capsule of claim 8 wherein the solubilizer is corn oil.

10. The capsule of claim 1 wherein the film-forming polymer is of natural origin.

11. The capsule of claim 10 wherein the film forming polymer is a natural film forming material selected from the group consisting of gelatin, shellac, alginates, pectin, and zeins.

12. The capsule of claim 11 wherein the natural film-forming polymer is gelatin.

13. The capsule of claim 1 wherein the film forming polymer is of synthetic origin.

14. The capsule of claim 13 wherein the film-forming polymer is selected from the group consisting of hydroxypropyl methyl cellulose, methyl cellulose, hydroxypropyl methyl cellulose acetate succinate, hydroxypropyl methyl cellulose phthalate, and cellulose acetate phthalate.

15. The capsule of claim 1 wherein the acid-insoluble polymer is selected from the group consisting of cellulose acetate phthalate, cellulose acetate butyrate, hydroxypropyl methyl cellulose phthalate, alginic acid salts such as sodium or potassium alginate, shellac, acrylic acid-methylacrylic acid copolymers.

16. The capsule of claim 15 wherein the acid-insoluble polymer is an acrylic acid-methacrylic acid copolymer.

17. The capsule of claim 1 wherein the acid-insoluble polymer is present in an amount from about 8 to about 20% by weight of the wet gelatin mass.

18. The capsule of claim 17 wherein the acid-insoluble polymer is present in an amount of about 12% by weight of the wet gelatin mass.

19. The capsule of claim 1 wherein the weight ratio of acid-insoluble polymer to film-forming polymer is from about 15% to about 50%.

20. The capsule of claim 1 wherein the aqueous solvent is water

21. The capsule of claim 1 wherein the aqueous solvent an aqueous solution of an alkali selected from the group consisting of ammonia, sodium hydroxide, potassium hydroxide, hydroxyl amine, triethanol amine, and ethylene diamine.

22. The capsule of claim 21 wherein the aqueous solvent is present in an amount sufficient to give a final pH of the gelatin mass of less than or equal to 9.0.

23. The capsule of claim 21 wherein the aqueous solvent is present in an amount sufficient to give a final pH of the gelatin mass of less than or equal to 8.5.

24. The capsule of claim 20 wherein the aqueous solvent is present in an amount sufficient to give a final pH of the gelatin mass of less than or equal to 8.0.

25. The capsule of claim 1 wherein the plasticizer to polymer ratio is from about 10% to about 50% of the polymer weight.

26. The capsule of claim 1 wherein the final moisture content of the capsule is from about 2% to about 10% by weight of the capsule.

27. The capsule of claim 26 wherein the final moisture content of the capsule is from about 4% to about 8% by weight of the capsule.

28. A capsule comprising valproic acid, wherein the valproic acid is released following oral administration to a fasting individual to produce a Cₘₐₓ between approximately 37.6 and 72.5 mg valproic acid/ml blood with a Tₘₐₓ of between 1 and 4 hours.

29. The capsule of claim 28 wherein the valproic acid is released following oral administration to a non-fasting individual to produce a Cₘₐₓ between 27.2 and 58.64 mg valproic acid/ml blood with a Tₘₐₓ of between 3 and 9 hours.

30. The capsule of claim 28 wherein the Cₘₐₓ is between 42.3 and 67.5 mg valproic acid/ml blood with a Tₘₐₓ of between 1.35 and 3 hours in a fasting individual.

31. The capsule of claim 29 wherein the Cₘₐₓ is between 31 and 53.8 mg valproic acid/ml blood with a Tₘₐₓ of between 3 and 9 hours.
